(19) United States
(12) Patent Application Publication
(10) Pub. No.: US 2022/0395342 A1
(43) Pub. Date: Dec. 15, 2022

(54) MULTI-ARM ROBOTIC SYSTEMS AND METHODS FOR MONITORING A TARGET OR PERFORMING A SURGICAL PROCEDURE

(71) Applicant: Mazor Robotics Ltd., Caesarea (IL)

(72) Inventors: Noam Weiss, Haifa (IL); Yizhaq Shmayahu, Ramat HaSharon (IL)

(21) Appl. No.: 17/344,658

(22) Filed: Jun. 10, 2021

(51) Int. Cl. A61B 34/37; A61B 8/00; A61B 6/00; A61B 34/20

(52) U.S. Cl. CPC A61B 34/37; A61B 8/4218; A61B 6/4458; A61B 34/20; A61B 2034/107

(57) ABSTRACT

Multi-arm robotic systems and methods for monitoring a target are provided. The system may include a first robotic arm configured to orient a first component and a second robotic arm configured to orient a second component. The first robotic arm and the second robotic arm may be co-registered. The first robotic arm may be caused to orient the first component at a first pose. The second robotic arm may be caused to orient the second component at a second pose. At least one image may be received from the first component and the second component.

### FIELD

The present technology generally relates to robotic systems using multiple robotic arms, and relates more particularly to using co-registered robotic arms to monitor a target and/or perform a surgical procedure.

### BACKGROUND

Surgical robots may be used to hold one or more imaging devices, tools, or devices during a surgery, and may operate autonomously (e.g., without any human input during operation), semi-autonomously (e.g., with some human input during operation), or non-autonomously (e.g., only as directed by human input).

### SUMMARY

Example aspects of the present disclosure include:

A system for imaging a target according to at least one embodiment of the present disclosure comprises a first robotic arm configured to orient a first component; a second robotic arm configured to orient a second component; at least one processor; and a memory storing data for processing by the processor, the data, when processed, causing the processor to: co-registering the first robotic arm and the second robotic arm; cause the first robotic arm to orient the first component at a first pose; cause the second robotic arm to orient the second component at a second pose; and receive at least one image from the first component and the second component.

Any of the aspects herein, wherein the memory stores further data for processing by the processor that, when processed, causes the processor to: receive a surgical plan, the surgical plan including the first pose.

Any of the aspects herein, wherein the first component comprises a source of at least one of an x-ray device and an ultrasound device, and the second component comprises a detector of at least one of the x-ray device and the ultrasound device.

Any of the aspects herein, wherein the memory stores further data for processing by the processor that, when processed, causes the processor to: determine the second pose based on the first pose.

Any of the aspects herein, wherein the second pose is the same as the first pose.

A system for performing a surgical procedure according to at least one embodiment of the present disclosure comprises a first robotic arm configured to orient a tool; a second robotic arm configured to orient an imaging device; at least one processor; and a memory storing data for processing by the processor, the data, when processed, causing the processor to: co-registering the first robotic arm and the second robotic arm; cause the second robotic arm to orient the imaging device at a target; cause the imaging device to monitor the target; and cause the first robotic arm to perform the surgical procedure using the tool.

Any of the aspects herein, wherein the memory stores further data for processing by the processor that, when processed, causes the processor to: receive an image depicting the target; and processing the image to identify the target.

Any of the aspects herein, wherein the surgical procedure is at least one of a biopsy, a decompression procedure, an amniocentesis procedure, and an ablation procedure.

Any of the aspects herein, wherein the target is at least one of one or more blood vessels, one or more nerves, electrical signals in one or more nerves, an organ, soft tissue, and hard tissue.

Any of the aspects herein, wherein the memory stores further data for processing by the processor that, when processed, causes the processor to: generate a notification when at least one of movement of the target and a change to the target is detected.

Any of the aspects herein, wherein the target is within a field of view of the imaging device and the tool is not within the field of view.

Any of the aspects herein, wherein the target is within a field of view of the imaging device and the tool is within the field of view.

Any of the aspects herein, wherein the memory stores further data for processing by the processor that, when processed, causes the processor to: generate a notification when at least a portion of the target is no longer within a field of view.

A system for performing one or more surgical tasks according to at least one embodiment of the present disclosure comprises a first robotic arm configured to orient a first tool; a second robotic arm configured to orient a second tool; at least one processor; and a memory storing data for processing by the processor, the data, when processed, causing the processor to: co-registering the first robotic arm and the second robotic arm; cause the first robotic arm to perform a first task using the first tool; and cause the second robotic arm to perform a second task using the second tool.

Any of the aspects herein, wherein the second task is dependent on the first task.

Any of the aspects herein, wherein the second task is independent of the first task.

Any of the aspects herein, wherein the first task is performed on a first anatomical element and the second task is performed on a second anatomical element.

Any of the aspects herein, wherein the first task and the second task is performed on an anatomical element.

Any of the aspects herein, wherein the first tool is the same as the second tool.

Any of the aspects herein, wherein the first tool is different than the second tool.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as Xₗ-Xₙ, Yₗ-Yₘ, and Zₗ-Zₒ, the phrase is intended to refer to a single element selected from X, Y, and Z, a combination of elements selected from the same class (e.g., X₁ and X₂) as well as a combination of elements selected from two or more classes (e.g., Yₗ and Zₒ).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a diagram of a system according to at least one embodiment of the present disclosure;

FIG. 3 is a flowchart of a method according to at least one embodiment of the present disclosure;

FIG. 4 is a diagram of a system according to at least one embodiment of the present disclosure;

FIG. 5 is an image from an example use case using the system of FIG. 4;

FIG. 6 is a flowchart of a method according to at least one embodiment of the present disclosure;

FIG. 7 is a flowchart of a method according to at least one embodiment of the present disclosure;

FIG. 8 is a diagram of a system according to at least one embodiment of the present disclosure; and

FIG. 9 is a flowchart of a method according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

By using co-registered robotic arms, multiple clinical applications may be performed that may utilize the combination of, for example, one of the following options:

(A) Two or more imaging arms. For example, in x-ray imaging, one robotic arm may hold a source and another robotic arm may hold a detector. In another example, in ultrasonic Time of Flight imaging, one robotic arm may hold a “source” probe, and the other robotic arm may hold a “detector” probe (both ultrasonic transducers). Another example may be a combination of ultrasonic imaging and optical coherent tomography imaging, which may use more than two robotic arms. By using the advantage of the co-registration, the two robotic arms of the X-ray imaging, for example, can be positioned, or even moved, very accurately opposite each other.

(B) At least one imaging arm and at least one operative arm. For example, a robotic arm supporting an imaging device can provide information to guide another robotic arm supporting an operative tool, either by imaging the tool or by imaging the area/volume that does not necessarily include the tool or the robotic arm supporting the tool. When imaging the operative tool, a pose of the operative tool can be compared to a pose of the operative tool as determined from the robotic arm to confirm an accuracy of the pose of the operative tool. The imaging device may also be useful when imaging an area without the operative tool, such as, for example, of a critical clinical object (such as, for example, a blood vessel or nerve). The imaging device may monitor the critical object to ensure that the operative tool does not reach it, for example, and thereby increasing safety of the procedure. In other examples, the co-registered arms may help move one robotic arm, according to an algorithm, based on a live imaging feedback from an imaging device held by another robotic arm.

(C) Two or more operative (e.g., effector) arms. In some embodiments, one robotic arm may support a first tool and another robotic arm may support a second tool, and the two robotic arms may work simultaneously on different parts of the body. For example, the two robotic arms may simultaneously drill screws in different vertebras. Because the robotic arms are co-registered, the robotic arms can avoid collision between the robotic arms and/or the tools they hold. In another example, one tool may rely on the other tool, for example, when inserting a specific tool, through another tool (e.g., a needle/screw through a guiding tube).

Embodiments of the present disclosure provide technical solutions to the problems of (1) operating two or more robotic arms while avoiding undesired contact; (2) monitoring a target with an imaging device while performing a surgical task or procedure; (3) performing two or more surgical tasks simultaneously or sequentially; and (4) increasing patient safety.

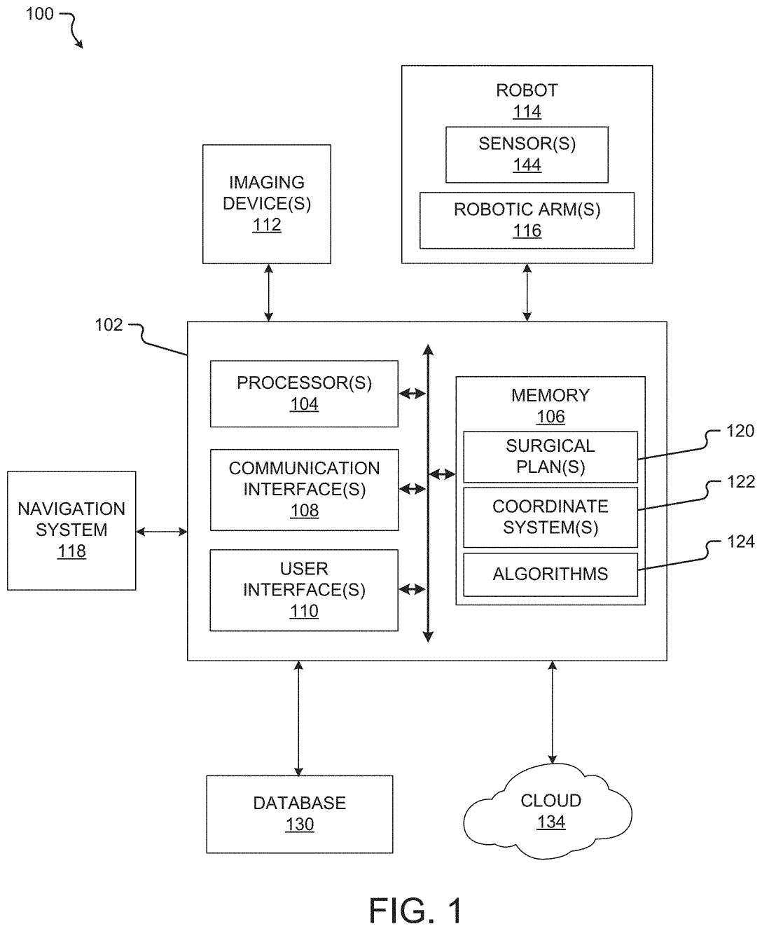

Turning first to FIG. 1, a block diagram of a system 100 according to at least one embodiment of the present disclosure is shown. The system 100 may be used to operate one or more robotic arms in a common coordinate system and/or carry out one or more other aspects of one or more of the methods disclosed herein. The system 100 comprises a computing device 102, one or more imaging devices 112, a robot 114 (which may further comprise one or more robotic arms 116 and/or one or more sensors 144), a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 300, 600, 700, and/or 900 described herein, or of any other methods. The memory 106 may store, for example, one or more surgical plan(s) 120, information about one or more coordinate system(s) 122 (e.g., information about a robotic coordinate system or space corresponding to the robot 114, information about a navigation coordinate system or space, information about a patient coordinate system or space), and/or one or more algorithms 124. Such algorithms may, in some embodiments, be organized into one or more applications, modules, packages, layers, or engines. Alternatively or additionally, the memory 106 may store other types of data (e.g., machine learning modes, artificial neural networks, etc.) or instructions that can be processed by the processor 104 to carry out the various method and features described herein. Thus, although various components of memory 106 are described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the sensor 144, the navigation system 118, the database 130, the cloud 134, and/or any other system or component not part of the system 100), and/or for transmitting instructions, images, or other information to an external system or device (e.g., another computing device 102, the imaging device 112, the robot 114, the sensor 144, the navigation system 118, the database 130, the cloud 134, and/or any other system or component not part of the system 100). The communication interface 108 may comprise one or more wired interfaces (e.g., a USB port, an ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (Mill) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise the sensors 144 that enable the processor 104 (or a processor of the robot 114) to determine a precise pose in space of the robotic arm (as well as any object or element held by or secured to the robotic arm).

In some embodiments, reference markers (i.e., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system may comprise one or more electromagnetic sensors. In various embodiments, the navigation system 118 may be used to track a position and orientation (i.e., pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system 122 to another (e.g., one or more robotic coordinate systems to a patient coordinate system and/or to a navigation coordinate system). The database 130 may additionally or alternatively store, for example, one or more surgical plans 120 (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 300, 600, 700, and/or 900 described herein. The system 100 or similar systems may also be used for other purposes.

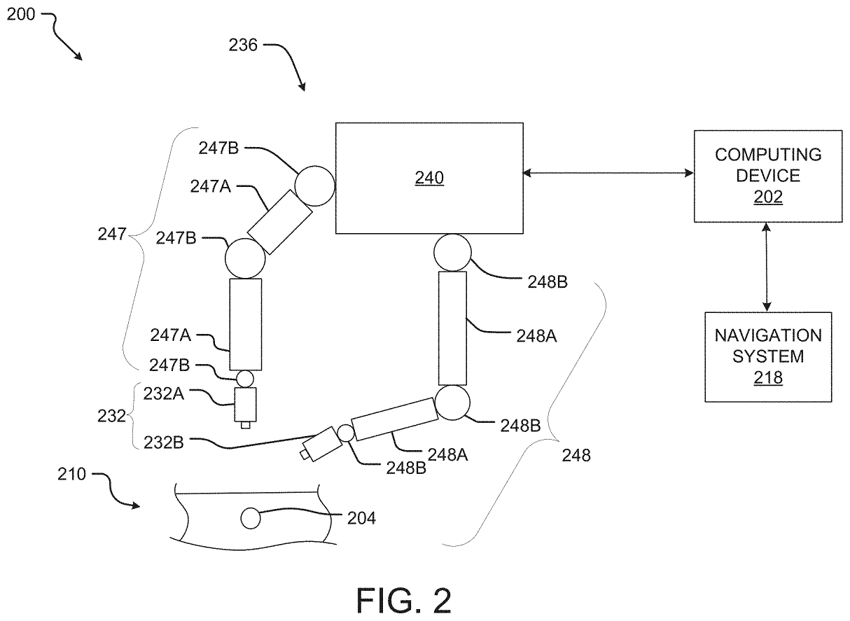

Turning to FIG. 2 a block diagram of a system 200 according to at least one embodiment of the present disclosure is shown. The system 200 includes a computing device 202 (which may be the same as or similar to the computing device 102 described above), a navigation system 218 (which may be the same as or similar to the navigation system 118 described above), and a robot 236 (which may be the same as or similar to the robot 114 described above). The system 200 may be used with the system 100 in some embodiments. Systems according to other embodiments of the present disclosure may comprise more or fewer components than the system 200. For example, the system 200 may not include the navigation system 218.

As illustrated, the robot 236 includes a first robotic arm 247 (which may comprise one or more members 247A connected by one or more joints 247B) and a second robotic arm 248 (which may comprise one or more members 248A connected by one or more joints 248B), each extending from a base 240. In other embodiments, the robot 236 may include one robotic arm or two or more robotic arms. The base 240 may be stationary or movable. The first robotic arm 247 and the second robotic arm 248 may operate in a shared or common coordinate space. By operating in the common coordinate space, the first robotic arm 247 and the second robotic arm 248 avoid colliding with each other during use, as a position of each robotic arm 247, 248 is known to each other. In other words, because each of the first robotic arm 247 and the second robotic arm 248 have a known position in the same common coordinate space, collision can be automatically avoided as a controller of the first robotic arm 247 and of the second robotic arm 248 is aware of a position of both of the robotic arms.

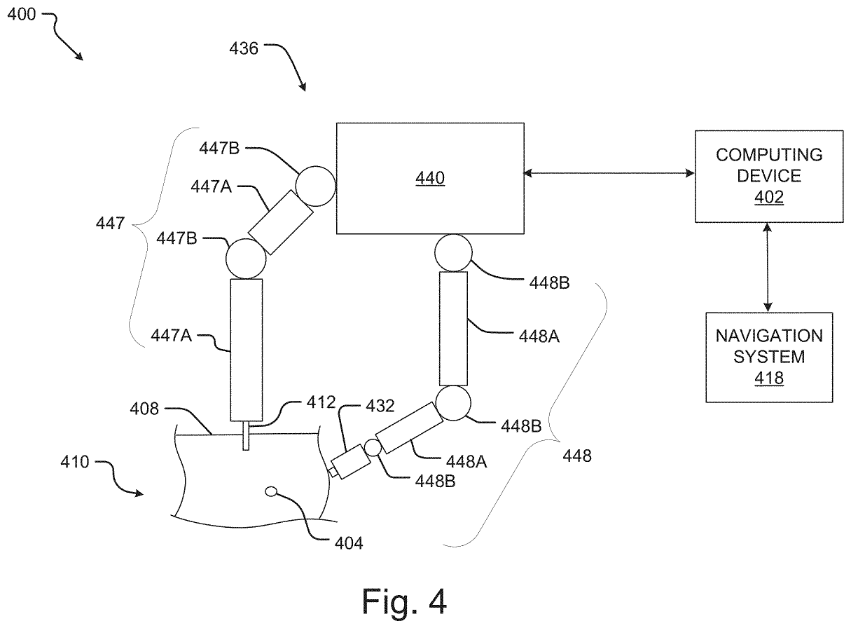

In some embodiments, one or more imaging devices or components 232 (which may be the same as or similar to the imaging device 112 described above) may be disposed or supported on an end of the first robotic arm 247 and/or the second robotic arm 248. In other embodiments, the imaging devices or components 232 may be disposed or secured to any portion of the first robotic arm 247 and/or the second robotic arm 248. In other embodiments, one or more tools 202 or instruments may be disposed on an end of each of the first robotic arm 247 and the second robotic arm 248 (as will be described with respect to FIGS. 4-9), though the tools or instruments may be disposed on any portion of the first robotic arm 247 and/or the second robotic arm 248. In further embodiments, both a tool 212 and an imaging device 232 may be supported on the same robotic arm 247 (as will be described with respect to FIGS. 4-7). In still other embodiments, any one or more tool(s), instrument(s), or imaging device(s) may be supported by, secured to, or disposed on a robotic arm. The first robotic arm 247 and/or the second arm 248 is operable to execute one or more planned movements and/or procedures autonomously and/or based on input from a surgeon or user.

As illustrated in FIG. 2, a first component 232A is supported by the first robotic arm 247 and a second component 232B is supported by the second robotic arm 248. It will be appreciated that co-registration of the first robotic arm 247 and the second robotic arm 248 enables the first robotic arm 247 to orient and operate the first component 232A and the second robotic arm 248 to orient and operate the second component 248B simultaneously or sequentially without collision or unintended contact.

In some embodiments, the first component 232A is a first imaging device and the second component 232B is a second imaging device. The first imaging device may be the same type of imaging device as the second imaging device. In other instances, the first imaging device may be a different type of imaging device than the second imaging device. As will be described in more detail below, the first imaging device and the second imaging device may each obtain one or more images of a target 204.

In other embodiments, the first component 232A may comprise a source of an imaging device, such as, for example, an ultrasound device or an x-ray device and the second component 232B may comprise a detector of the imaging device, which may be, for example, the ultrasound device or the x-ray device. The first component 232A and the second component 232B may be used to obtain one or more images of the target 204.

In the illustrated example, the target 204 is an anatomical element of a patient 210. In other embodiments, the target 204 may be an object, an incision, a tool, an instrument, a robotic arm, any component of the system 200, any component external to the system 200, or the like. In some embodiments, the one or more images combined with pose information of each of the imaging devices 232 may be used to determine a pose of the target 204. The pose information may be used to track movement of the target 204, as will be described further below. For example, the pose of the target 204 may be compared at different time increments to determine if the target 204 has moved. In other embodiments, additional image(s) of the target 204 may be taken from different angles by either the first component 232A or the second component 232B, or both, to determine a boundary of the target 204 and/or to update a pose of the object or the target 204 (for example, to update the pose because the target 204 has moved).

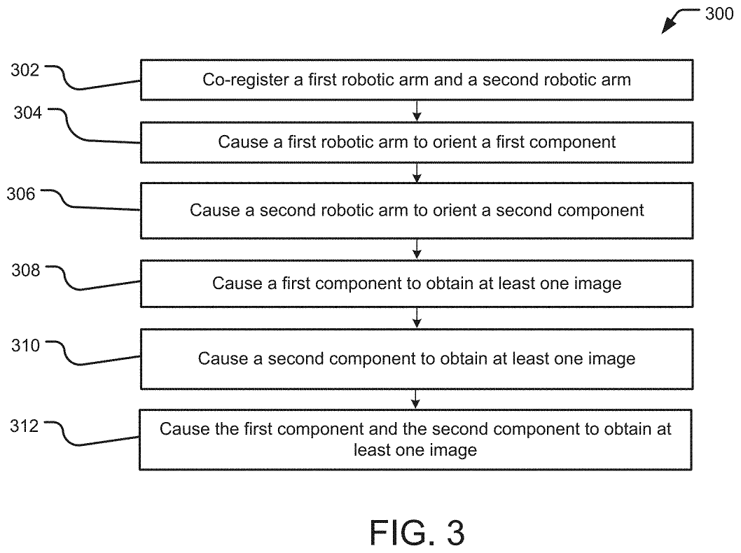

FIG. 3 depicts a method 300 that may be used, for example, for obtaining one or more images.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing instructions stored in a memory such as the memory 106. The instructions may correspond to one or more steps of the method 300 described below. The instructions may cause the processor to execute one or more algorithms, such as the algorithms 124.

The method 300 comprises co-registering a first robotic arm and a second robotic arm (step 302). A processor such as the processor 104 may execute an algorithm such as the algorithm 124 to co-register the first robotic arm and the second robotic arm. The first robotic arm may be the same as or similar to the first robotic arm 114, 247 and the second robotic arm may be the same as or similar to the second robotic arm 114, 248. The co-registering enables control of the first robotic arm and the second robotic arm in a common coordinate system so as to avoid undesired contact between the first robotic arm and the second robotic arm, and thus also to avoid undesired contact between end effectors of the first robotic arm and the second robotic arm. In some embodiments, the first robotic arm may be configured to support and/or orient a first component such as the first component 232A and the second robotic arm may be configured to support and/or orient a second component such as the second component 232B. Though in other embodiments, the first robotic arm and/or the second robotic arm may support and/or orient any tool, instrument, or imaging device.

In some embodiments, a computing device, such as the computing device 102, 202 computes and controls a pose of the first robotic arm and a pose of the second robotic arm. The pose of each robotic arm is known to the computing device, such that the computing device correlates the poses of the first robotic arm and the second robotic arm with respect to each other, and if desired, with respect to a preoperative image or preoperative image set. Intraoperatively, the poses of the first robotic arm and the second robotic arm may be updated in real-time and recorded by the computing device, based on the images provided to the system by the imaging device during the course of the procedure. The correlation of the coordinate systems enables a surgical procedure to be carried out with a higher degree of accuracy compared to a procedure carried out in which the two robotic arms are independently operated.

The method 300 also comprises causing a first robotic arm to orient the first component (step 304). The first robotic arm may orient the first component at one or more poses. In some embodiments, the one or more poses may be based on one or more steps from a surgical plan such as the surgical plan 120. In other embodiments, the one or more poses may be based on input received from a user via a user interface such as the user interface 110.

In some embodiments, instructions may be generated and transmitted to the first robotic arm to cause the first robotic arm to orient the first component at the one or more poses. Instructions may also be communicated to a user via the user interface to guide the user (whether manually or robotically assisted) to orient the first imaging device.

The method 300 also comprises causing a second robotic arm to orient a second component (step 306). The step 306 may the same as or similar to step 304 as applied to the second robotic arm. The second robotic arm may be the same as or similar to the second robotic arm 116, 248. In some embodiments, the second robotic arm may orient the second component at one or more poses that are different from the one or more poses of the first component. In other embodiments, the second robotic arm may orient the second component at one or more poses that are the same as the one or more poses of the first component. In such embodiments, the second component may be oriented at the same poses after the first component, or vice versa.

It will be appreciated that steps 304 and 306 may occur simultaneously or sequentially. It will also be appreciated that step 306 may occur independently of step 304 or may depend on step 304 (and vice versa). In other words, the second robotic arm may orient the second component based on the pose of the first component, or vice versa. For example, the first component may comprise a source of an ultrasound device or an x-ray device and the second component may comprise a detector of the ultrasound device or the x-ray device. In the same example, the second robotic arm may orient the detector based on a pose of the source.

The method 300 also comprises causing the first component to obtain at least one first image (step 308). In some embodiments, the first component comprises a first imaging device which may be the same as or similar to the imaging device 112, 232. The first image may comprise one or more 2D images, one or more 3D images, or a combination of one or more 2D images and one or more 3D images.

The first image may depict at least one target. The at least one target may be a reference marker, a marking on a patient anatomy, an anatomical element, an incision, a tool, an instrument, an implant, or any other object. The first image may be processed using an algorithm such as the algorithm 124 to process the image and identify the at least one target in the first image. In some embodiments, feature recognition may be used to identify a feature of the at least one target. For example, a contour of a screw, tool, edge, instrument, or anatomical element may be identified in the first image. In other embodiments, an image processing algorithm may be based on artificial intelligence or machine learning. In such embodiments, a plurality of training images may be provided to the processor, and each training image may be annotated to include identifying information about a target in the image. The processor, executing instructions stored in memory such as the memory 106 or in another memory, may analyze the images using a machine-learning algorithm and, based on the analysis, generate one or more image processing algorithms for identifying target(s) in an image. Such image processing algorithms may then be applied to the first image.

The method 300 also comprises causing the second component to obtain at least one second image (step 310). The step 304 may be the same as or similar to step 302 of method 300 described above with respect to obtaining the second image. The second component may be a second imaging device which may be the same as or similar to the imaging device 112, 232.

In some embodiments, the first imaging device may be, for example, an imaging device that obtains images using a first source, such as X-rays, and the second imaging device may be, for example, an imaging device that obtains images using a second source, such as ultrasound. In such embodiments, when the first imaging device and the second imaging device image the same anatomical feature (whether from the same or different poses), images obtained from the second imaging device may supplement or provide additional information to images obtained from the first imaging device. For example, images from an ultrasound device may provide soft tissue information that can be combined with images from an x-ray device that may provide hard tissue information.

In other embodiments, the first imaging device may be the same as the second imaging device. In at least some embodiments, the first imaging device may image a different anatomical feature or area of the patient than the second imaging device. In other embodiments, the first imaging device may image the same anatomical feature or area of the patient as the second imaging device.

It will be appreciated that in some embodiments, the method 300 may not include the steps 308 and/or 310.

The method 300 also comprises causing the first component and the second component to obtain at least one image (step 312). It will be appreciated that step 312 may be executed independent of claims 308 and 310. It will be appreciated that in some embodiments, the method 300 may not include the step 312.

In some embodiments, the first component is a source of an ultrasound or an x-ray device and the second component is a detector of the ultrasound or the x-ray device. In such embodiments, the detector may be oriented based on a pose of the source or vice versa. The at least one image may be obtained from, for example, the detector detecting the source waves (whether ultrasound, x-ray, or any other wavelength) emitted by the source.

It will be appreciated that the method 300 may be executed with more than two robotic arms. For example, the method 300 may cause a third robotic arm to orient a third imaging device and obtain an image from the third imaging device. It will also be appreciated, that the method 300 may be executed with any number of robotic arms and/or imaging devices. For example, the method 300 may cause the second robotic arm to orient the second and third imaging devices.

The present disclosure encompasses embodiments of the method 300 that comprise more or fewer steps than those described above, and/or one or more steps that are different than the steps described above.

Turning to FIG. 4, a block diagram of a system 400 according to at least one embodiment of the present disclosure is shown. The system 400 includes a computing device 402 (which may be the same as or similar to the computing device 102 described above), a navigation system 418 (which may be the same as or similar to the navigation system 118 described above), and a robot 436 (which may be the same as or similar to the robot 114, 236 described above). Systems according to other embodiments of the present disclosure may comprise more or fewer components than the system 400. For example, the system 400 may not include the navigation system 418.

As illustrated, the robot 436 includes a first robotic arm 447 (which may comprise one or more members 447A connected by one or more joints 447B) and a second robotic arm 448 (which may comprise one or more members 448A connected by one or more joints 448B), each extending from a base 440. In other embodiments, the robot 436 may include one robotic arm or two or more robotic arms. The base 440 may be stationary or movable. The first robotic arm 447 and the second robotic arm 448 may operate in a shared or common coordinate space. By operating in the common coordinate space, the first robotic arm 447 and the second robotic arm 448 avoid colliding with each other during use, as a position of each robotic arm 447, 448 is known to each other. In other words, because each of the first robotic arm 447 and the second robotic arm 448 have a known position in the same common coordinate space, collision can be automatically avoided as a controller of the first robotic arm 447 and of the second robotic arm 448 is aware of a position of both of the robotic arms.

As illustrated in FIG. 4, a tool 412 is supported by the first robotic arm 447 and an imaging device 432 is supported by the second robotic arm 448. The imaging device 432 may be the same as or similar to the imaging device 112, 232 or any other imaging device described herein. The tool 412 may be used to perform an action or a procedure on a patient 410, whether based on instructions from a surgeon and/or pursuant to a surgical plan such as the surgical plan 120. For example, the tool 412 may be used to form an incision on a surface 408 of the patient 410, perform an ablation, perform an amniocentesis procedure, perform a biopsy, or any other surgical task or procedure.

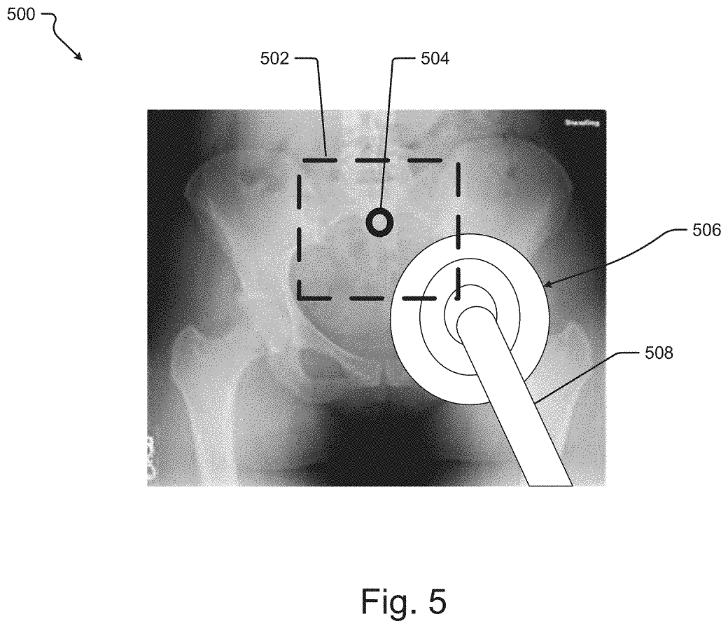

In some embodiments, the imaging device 432 may be used to track and detect movement of a critical target 404. In at least one embodiment, the tool 412 may be in a field of view of the imaging device 432 when the imaging device 432 tracks the target 404. In other embodiments, the tool 412 may not be in the field of view of the imaging device 432 when the imaging device 432 tracks the target 404. It will be appreciated that because the first robotic arm 447 and the second robotic arm 448 are co-registered, and a pose of each robotic arm is known, that the imaging device 432 may track a target 404 without tracking the tool 412. In other words, the tool 412 may not be in the field of view of the imaging device 432. In some embodiments, the imaging device 432 may track or monitor the critical target 404 to prevent the tool 412 from, for example, damaging the target 404. For example, the imaging device 432 may track the target 404 to prevent, for example, heat from an ablation probe from damaging the target 404, as illustrated in FIG. 5.

In other embodiments, when movement is detected, a path of the tool 412 can be updated or adjusted. For example, the imaging device 432 may track an incision for movement. When movement of the incision is detected, a path of the tool 412 (that is outside of the incision) may be shifted to reach a position of the incision after movement.

In still other embodiments, the imaging device 432 may be used to track and/or guide movement of the tool 412. For example, the imaging device 432 may image the tool 412 and provide image data of the tool 412 and the area surrounding the tool 412 to a processor such as the processor 104. The processor can determine if the tool 412 may contact the target 404 and may update the tool path to avoid such target 404. The processor may also determine a pose of the tool 412, which may be compared to pose information obtained from the first robotic arm 447 to confirm an accuracy of the pose of the tool 412.

It will be appreciated that co-registration of the first robotic arm 447 and the second robotic arm 448 enables the first robotic arm 447 to orient and operate the tool 412 and the second robotic arm 448 to orient and operate the imaging device 432 simultaneously or sequentially without collision.

FIG. 5 depicts an example X-ray image 500 of a patient anatomy according to at least one embodiment of the present disclosure. In the image 500, an ablation procedure is shown. The procedure may use a system such as the system 400 to monitor a critical target 504 during the procedure. A tool 508 may be the same as or similar to the tool 412. In the illustrated example, the tool 508 may comprise an ablation probe.

In the illustrated embodiment, a field of view 502 of an imaging device such as the imaging device 112, 232, 432, is shown as a dotted rectangle; a critical target 504 (which may be the same as or similar to the target 404) is shown as a circle; and a penetration zone 506 of the tool 508 is shown as a series of circles for illustrative purposes. In the illustrated embodiment, the tool 508 may be an ablation probe and the penetration zone 506 may correlate to heat zones of the ablation tool. In other embodiments, the penetration zone 506 may simply be a tip of the tool 508 such as, for example, when the tool 508 is a needle.

In some embodiments, the target 504 may be identified prior to the surgical procedure. For example, the imaging device may image an area and transmit the image data to a processor such as the processor 104. The processor may automatically identify the target 504. In other examples, input may be received from a user, such as a surgeon, to identify the target 504.

The critical target 504 may be monitored by sending image data containing the field of view 502 to the processor. The processor may monitor for movement of the target 504, changes to the target 504, or changes to the field of view 502. A change to the field of view 502 may be, for example, a change in tissue within the field of view 502 that has been ablated by the ablation tool 508 or a change in tissue that has been cut by, for example, a knife. The change to the field of view 502 may indicate that the corresponding tool 508 causing the change may be approaching the target 504. When a change is detected, whether to the field of view 504 or the target 504, a notification may be generated and transmitted to a user such as a surgeon or other medical provider. In some embodiments, the notification may be generated when the change reaches a threshold. For example, the threshold may be the minimum distance allowed between the target 504 to the tool 508. In other examples, the notification may be generated when at least a portion of the target 504 is not within the field of view 502.

It will be appreciated that the tool 508 may not be within the field of view 502 in some embodiments, such as that shown in the illustrated embodiment. In other words, the imaging device may simply monitor the target 504 without monitoring the tool 508.

The target 504 may be any anatomical element or an anatomical area. For example, the target may be one or more blood vessels, one or more nerves, electrical signals in one or more nerves, an organ, soft tissue, or hard tissue. The target 504 may also be a boundary or border, whether a boundary of an anatomical element or a user defined boundary. For example, the target may be a border of a tumor or a nearby critical structure such as a nerve or vessel, which may be monitored while the tumor is ablated by an ablation probe.

It will be appreciated that though FIG. 5 shows an ablation procedure in which the system 400 may be used, any procedure may use the system 400 in the same manner to monitor a target while performing a surgical task or procedure. In some embodiments, the procedure may be any spinal or cranial surgical procedure. In other embodiments, the procedure may be any surgical procedure or task. Example procedures include a thyroid biopsy, a liver biopsy, a peripheral lung biopsy, a bone marrow aspiration and biopsy, or an arthrocentesis procedure.

In some embodiments, the procedure may be, for example, an amniocentesis, the target 504 may be a fetus and/or an amniotic sack wall, and the tool 508 may be a needle for removing amniotic fluids. In such procedures, a location and movement of the fetus may be monitored by an imaging device, for example, an ultrasonic probe. In at least some embodiments, a preliminary scan of the designated area for probing may be initially obtained from the imaging device. The fetus may then be identified by a user, such as, for example, a surgeon, or identified automatically by the processor using artificial intelligence. The processor may track movement of the fetus from additional image data that may be received from the imaging device. As the fetus moves, the robotic arm may automatically reorient the imaging device to track the fetus. In some embodiments, the amniotic sack wall can also be identified by the user or identified automatically by the processor using artificial intelligence. During the procedure, the fetus and/or the amniotic sack wall may be monitored to prevent damage from the needle to the fetus and/or the amniotic sack wall.

In other embodiments, the procedure may be a flavectomy, the target 504 may be a dural sac, and the tool 508 may be any tool to perform the flavectomy. In at least some of the embodiments, a ligamentum flavum may be imaged by the imaging device (which may be, for example, an ultrasound probe), the dural sac may be identified in the image data, and the dural sac may be monitored while the flavectomy is performed.

In still other embodiments, the procedure may be a decompression procedure, the target 504 may be a nerve, and the tool 508 may be any tool to perform the decompression procedure. In at least some of the embodiments, the target 504 may include the structure surrounding the nerve. The structure and/or the nerve may also be monitored to determine and/or confirm that the nerve is free from decompression after the decompression procedure has been executed.

In other embodiments, the target may by a patient, and the patient may be monitored for undesired movement.

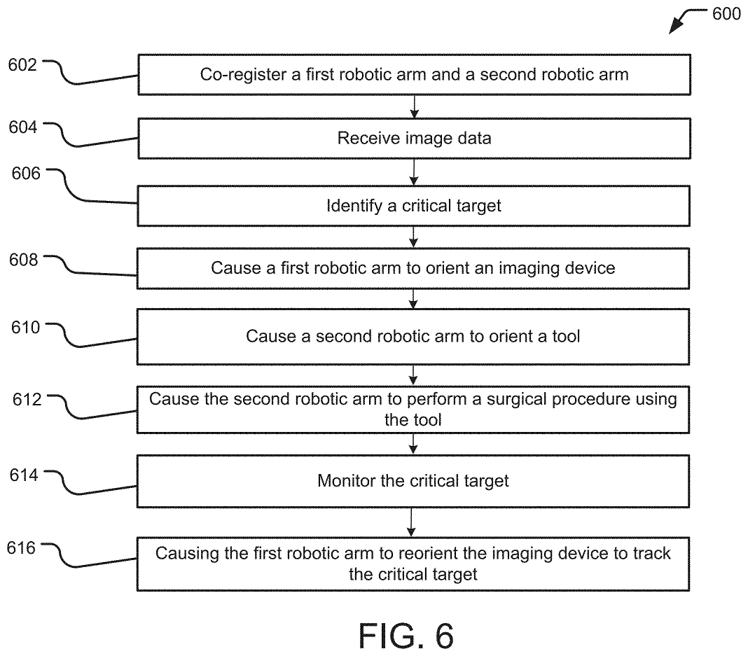

FIG. 6 depicts a method 600 that may be used, for example, monitoring a critical target and performing a surgical procedure or task.

The method 600 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 600. The at least one processor may perform the method 600 by executing instructions stored in a memory such as the memory 106. The instructions may correspond to one or more steps of the method 600 described below. The instructions may cause the processor to execute one or more algorithms, such as the algorithms 124.

The method 600 comprises co-registering a first robotic arm and a second robotic arm (step 602). The step 602 may be the same as or similar to step 302 of method 300. The first robotic arm may be the same as or similar to the first robotic arm 116, 247, 447. The second robotic arm may be the same as or similar to the second robotic arm 116, 248, 448.

The method 600 also comprises receiving an image (step 604). The image may comprise one or more 2D images, one or more 3D images, or a combination of one or more 2D images and one or more 3D images. In some embodiments, the image may be received from an imaging device such as the imaging device 112, 232, 432. In other embodiments, the image may be received via a user interface such as the user interface 110 and/or via a communication interface such as the communication interface 108 of a computing device such as the computing device 102 or 202, and may be stored in a memory such as the memory 106. The image may also be generated by and/or uploaded to any other component of the system 100, 200, or 400. In some embodiments, the image may be indirectly received via any other component of the system or a node of a network to which the system is connected.

The image may depict a critical target, described in more detail below. The critical target may be, for example, an anatomical element, an anatomical area, an instrument, a tool, a boundary or border, one or more nerves, one or more blood vessels, a dural sac, a fetus, an amniotic sack, or electrical signals in one or more nerves.

The method 600 also comprises identifying the critical target (step 606). The image may be processed using an algorithm such as the algorithm 124 to process the image and identify the critical target in the image. In some embodiments, feature recognition may be used to identify a feature of the target. For example, a contour of a screw, port, tool, edge, instrument, or anatomical element may be identified in the first image. In other embodiments, an image processing algorithm may be based on artificial intelligence or machine learning. In such embodiments, a plurality of training images may be provided to the processor, and each training image may be annotated to include identifying information about a target in the image. The processor, executing instructions stored in memory such as the memory 106 or in another memory, may analyze the images using a machine-learning algorithm and, based on the analysis, generate one or more image processing algorithms for identifying target(s) in an image. Such image processing algorithms may then be applied to the image received.

The method 600 also comprises causing a first robotic arm to orient the imaging device (step 608). The step 608 may be the same as or similar to step 304 of method 300 with respect to orienting the imaging device. Additionally, the first robotic arm may be instructed to orient the imaging device at the critical target.

The method 600 also comprises causing a second robotic arm to orient a tool (step 610). The step 610 may be the same as or similar to step 304 of method 300 as applied to the second robotic arm orienting the tool. The tool may be the same as or similar to the tool 412, 508.

The method 600 also comprises causing the second robotic arm to perform a surgical procedure using the tool (step 612). In some embodiments, instructions may be generated and transmitted to the second robotic arm to cause the second robotic arm to orient and/or operate the first tool. Instructions may also be communicated to a user via a user interface such as the user interface 112 to guide a user (whether manually or robotically assisted) to orient and/or operate the tool.

The procedure may be any surgical procedure or task. The procedure may be, for example, an ablation procedure, a decompression procedure, an amniocentesis procedure, a flavectomy, a biopsy, a thyroid biopsy, a liver biopsy, a peripheral lung biopsy, a bone marrow biopsy, or an arthrocentesis procedure.

The method 600 also comprises monitoring the critical target using the imaging device (step 614). Monitoring the critical target may comprise causing the imaging device to transmit image data to a processor such as the processor 104. The image data may depict a field of view of the imaging device. The critical target may be within a field of view of the imaging device. In some embodiments, the tool may be within the field of view. In other embodiments, the tool may not be in a field of view of the imaging device, as described with respect to, for example, FIG. 5.

The processor may receive the image data depicting the target and monitor the target for changes to the target, movement of the target, or a change to the field of view. In some other embodiments, when the processor identifies a change in the target, the processor may generate a notification to the user to alert the user that the target has changed (whether, for example, the target has been affected by an ablation, a biopsy, a decompression procedure, or the like). In some embodiments, a notification may be generated when the change to the target has reached a threshold. For example, a notification may be generated if more than 10% of the target is affected by an ablation procedure.

The method 600 also comprises causing the first robotic arm to reorient the imaging device to track the critical target (step 616). In some embodiments, when the processor identifies movement of the target, such as, for example, in step 614, such that at least a portion of the target is no longer within a field of view of the imaging device, the processor may generate instructions to cause the first robotic arm to reorient the imaging device so that the target is completely within the field of view of the imaging device. In other embodiments, when the processor identifies movement of the target such that the portion of the target is no longer within the field of view of the imaging device, the processor may generate a notification to a user, such as a surgeon or other medical provider, that the target has moved.

The present disclosure encompasses embodiments of the method 600 that comprise more or fewer steps than those described above, and/or one or more steps that are different than the steps described above.

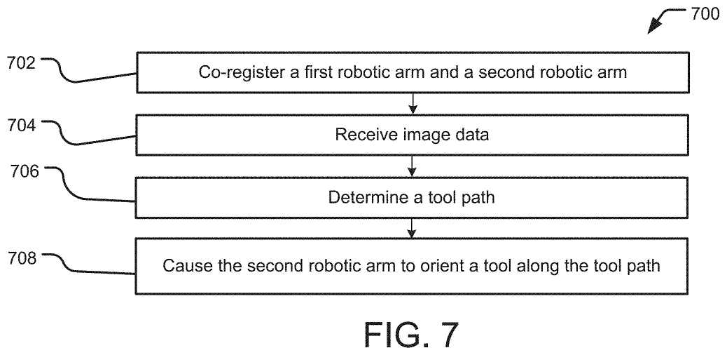

FIG. 7 depicts a method 700 that may be used, for example, for determining a tool path.

The method 700 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 700. The at least one processor may perform the method 700 by executing instructions stored in a memory such as the memory 106. The instructions may correspond to one or more steps of the method 700 described below. The instructions may cause the processor to execute one or more algorithms, such as the algorithms 124.

The method 700 comprises co-registering a first robotic arm and a second robotic arm (step 702). The step 702 may be the same as or similar to step 302 of method 300. The first robotic arm may be the same as or similar to the first robotic arm 116, 247, 447. The second robotic arm may be the same as or similar to the second robotic arm 116, 248, 448.

The method 700 also comprises receiving image data (step 704). The step 704 may be the same as or similar to step 604 of method 600. The image data may be received from an imaging device such as the imaging device 112, 232, 432.

The method 700 also comprises determining a tool path (step 706). The tool path may be based on, for example, one or more inputs such as the image data received in step 704, a surgical plan such as the surgical plan 120, or dimensions and/or functionality of a tool such as the tool 412, 508 selected for the surgical procedure or tasks. A processor such as the processor 104 may determine the tool path using the one or more inputs.

The method 700 also comprises causing the first robotic arm to orient a tool along the tool path (step 708). In some embodiments, the first robotic arm may orient an instrument or an implant along the tool path, or any other predetermined path. Instructions may be generated and/or transmitted to the first robotic arm to cause the first robotic arm to automatically orient the tool along the tool path. The instructions may also be displayed on a user interface such as the user interface 110 to instruct a user to guide the tool along the tool path (whether manually or robotically assisted). The tool path may be obtained from a surgical plan such as the surgical plan 120, may be input by a user via the user interface, and/or may be calculated prior to or during a surgical procedure such as, for example, in step 706.

The present disclosure encompasses embodiments of the method 700 that comprise more or fewer steps than those described above, and/or one or more steps that are different than the steps described above.

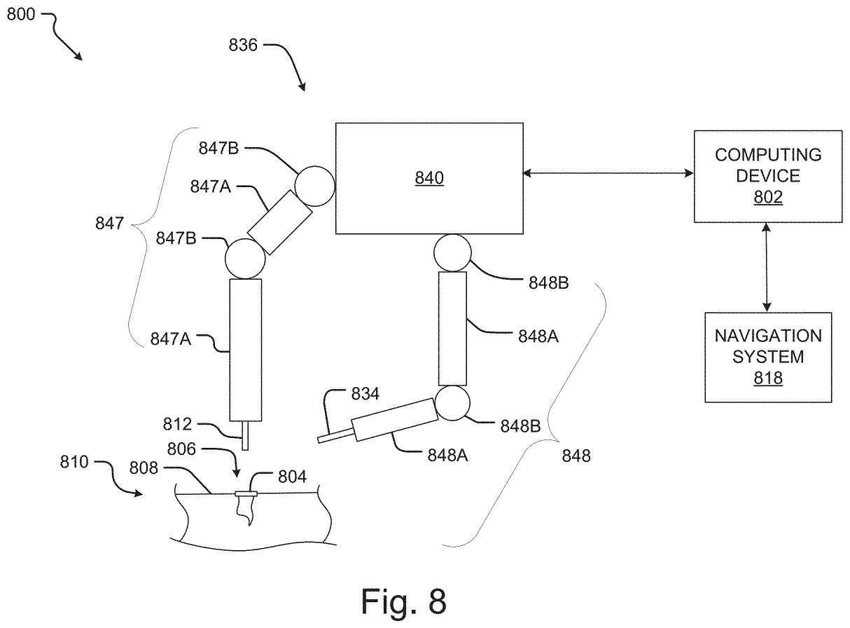

Turning to FIG. 8 a block diagram of a system 800 according to at least one embodiment of the present disclosure is shown. The system 800 includes a computing device 802 (which may be the same as or similar to the computing device 102 described above), a navigation system 818 (which may be the same as or similar to the navigation system 118 described above), and a robot 836 (which may be the same as or similar to the robot 114 described above). Systems according to other embodiments of the present disclosure may comprise more or fewer components than the system 800. For example, the system 800 may not include the navigation system 818.

As illustrated, the robot 836 includes a first robotic arm 847 (which may comprise one or more members 847A connected by one or more joints 847B) and a second robotic arm 848 (which may comprise one or more members 848A connected by one or more joints 848B), each extending from a base 840. In other embodiments, the robot 836 may include one robotic arm or two or more robotic arms. The base 840 may be stationary or movable. The first robotic arm 847 and the second robotic arm 848 may operate in a shared or common coordinate space. By operating in the common coordinate space, the first robotic arm 847 and the second robotic arm 848 avoid colliding with each other during use, as a position of each robotic arm 847, 848 is known to each other. In other words, because each of the first robotic arm 847 and the second robotic arm 848 have a known position in the same common coordinate space, collision can be automatically avoided as a controller of the first robotic arm 847 and of the second robotic arm 848 is aware of a position of both of the robotic arms.

As illustrated in FIG. 8, a first tool 812 is supported by the first robotic arm 848 and a second tool 834 is supported by the second robotic arm 848. The first tool 812 and the second tool 834 may each be the same as or similar to the tool 412, 508. In some embodiments, the first tool 812 is the same as the second tool 834. In other embodiments, the first tool 812 may be different than the second tool 834. Each of the first tool 812 and the second tool 834 may be used to perform an action or a procedure on a patient 810, whether based on instructions from a surgeon and/or pursuant to a surgical plan such as the surgical plan 120. For example, the first tool 812 may be used to form an incision 806 on a surface 808 of the patient 810 and the second tool 834 may insert a port 804.

In some embodiments, the first tool 812 may perform a first task and the second tool 834 may perform a second task based on the first task. For example, the first robotic arm 847 may insert a guiding tube (e.g., the first tool 812) and the second robotic arm 848 may insert a needle or a screw (e.g., the second tool 834) through the guiding tube. In other embodiments, the first tool 812 may perform the first task independent of the second tool 834 performing the second task. For example, the first robotic arm 847 may drill a first screw (e.g., the first tool 812) into a first vertebra and the second robotic arm 848 may drill a second screw (e.g., the second tool 824) into a second vertebra simultaneously.

It will be appreciated that co-registration of the first robotic arm 847 and the second robotic arm 848 enables the first robotic arm 847 to orient and operate the first tool 812 and the second robotic arm 848 to orient and operate the second tool 834 simultaneously or sequentially without collision.

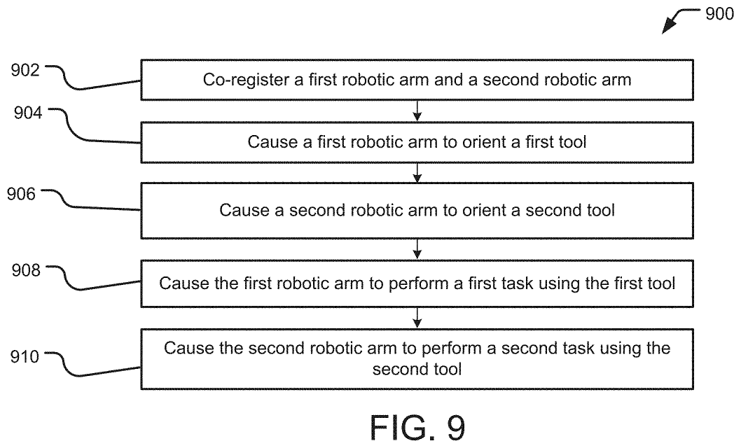

FIG. 9 depicts a method 900 that may be used, for example, performing one or more surgical procedures.

The method 900 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 900. The at least one processor may perform the method 900 by executing instructions stored in a memory such as the memory 106. The instructions may correspond to one or more steps of the method 900 described below. The instructions may cause the processor to execute one or more algorithms, such as the algorithms 124.

The method 900 comprises co-registering a first robotic arm and a second robotic arm (step 902). The step 902 may be the same as or similar to step 302 of method 300. The first robotic arm may be the same as or similar to the first robotic arm 116, 247, 447, 847. The second robotic arm may be the same as or similar to the second robotic arm 116, 248, 448, 848.

The method 900 also comprises causing a first robotic arm to orient a first tool (step 904). The step 904 may be the same as or similar to step 304 of method 300 as applied to the first robotic arm orienting the first tool. The first tool may be the same as or similar to the first tool 412, 508, 812.

The method 900 also comprises causing the second robotic arm to orient a second tool (step 906). The step 906 may be the same as or similar to step 304 of method 300 as applied to the second robotic arm orienting the second tool. The second tool may be the same as or similar to the first tool 412, 508, 834. In some embodiments, the second tool may be the same as the first tool. In other embodiments, the second tool may be different than the first tool.

The method 900 also comprises causing the first robotic arm to perform a first task using the first tool (step 908). The step 908 may be the same as or similar to step 612 of method 600. In some embodiments, the first task may be based on a step from a surgical plan such as the surgical plan 120. In other embodiments, the second task may be based on input received from a user such as a surgeon or other medical provider via a user interface such as the user interface 110.

The method 900 also comprises causing the second robotic arm to perform a second task using the second tool (step 910). The step 910 may be the same as or similar to step 612 of method 600. The second task may be based on a step from the surgical plan or input received from the user. In some embodiments, the second task may rely on or be dependent on the first task. For example, the first tool may be a guide tube and the first task may comprise insertion of the guide tube into an incision. In the same example, the second tool may be a needle and the second task may comprise inserting the needle into the guide tube. In other embodiments, the second task may be independent of the first task. For example, the first tool may be a first knife and the first task may comprise forming a first incision using the first knife. In the same example, the second tool may be a second knife and the second task may comprise forming a second incision using the second knife. In some embodiments, the first tool may perform a first task on a first anatomical element and the second tool may perform a second task on a second anatomical element different from the first anatomical element. For example, the first tool may be a first screw and the first task may comprise screwing the first screw into a first vertebra. In the same example, the second tool may be a second screw and the second task may comprise screwing the second screw into a second vertebra.

It will be appreciated that the method 900 may include orienting more than two tools (e.g., a third tool, a fourth tool, etc.) and/or performing more than two tasks (e.g., a third task, a fourth task, etc.). It will also be appreciated that additional tasks may be performed by any tool.

The present disclosure encompasses embodiments of the method 900 that comprise more or fewer steps than those described above, and/or one or more steps that are different than the steps described above.

It will be appreciated that the systems 200, 400, 800 and the methods 300, 600, 700, 900 may use more than two robotic arms. For example, a system may include three robotic arms and may comprise a first robotic arm supporting a first imaging device, a second robotic arm supporting a second imaging device, and a third robotic arm supporting a tool.

As noted above, the present disclosure encompasses methods with fewer than all of the steps identified in FIGS. 3, 6, 7, and 9 (and the corresponding description of the methods 300, 600, 700, and 900), as well as methods that include additional steps beyond those identified in FIGS. 3, 6, 7, and 9 (and the corresponding description of the methods 300, 600, 700, and 900). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for imaging a target comprising:
a first robotic arm configured to orient a first component;
a second robotic arm configured to orient a second component;
at least one processor; and
a memory storing data for processing by the processor, the data, when processed, causing the processor to:
co-registering the first robotic arm and the second robotic arm;
cause the first robotic arm to orient the first component at a first pose;
cause the second robotic arm to orient the second component at a second pose; and
receive at least one image from the first component and the second component.

2. The system of claim 1, wherein the memory stores further data for processing by the processor that, when processed, causes the processor to:
receive a surgical plan, the surgical plan including the first pose.

3. The system of claim 1, wherein the first component comprises a source of at least one of an x-ray device and an ultrasound device, and the second component comprises a detector of at least one of the x-ray device and the ultrasound device.

4. The system of claim 1, wherein the memory stores further data for processing by the processor that, when processed, causes the processor to:
determine the second pose based on the first pose.

5. The system of claim 1, wherein the second pose is the same as the first pose.

6. A system for performing a surgical procedure comprising:
a first robotic arm configured to orient a tool;
a second robotic arm configured to orient an imaging device;
at least one processor; and
a memory storing data for processing by the processor, the data, when processed, causing the processor to:
co-registering the first robotic arm and the second robotic arm;
cause the second robotic arm to orient the imaging device at a target;
cause the imaging device to monitor the target; and
cause the first robotic arm to perform the surgical procedure using the tool.

7. The system of claim 6, wherein the memory stores further data for processing by the processor that, when processed, causes the processor to:
receive an image depicting the target; and
processing the image to identify the target.

8. The system of claim 6, wherein the surgical procedure is at least one of a biopsy, a decompression procedure, an amniocentesis procedure, and an ablation procedure.

9. The system of claim 6, wherein the target is at least one of one or more blood vessels, one or more nerves, electrical signals in one or more nerves, an organ, soft tissue, and hard tissue.

10. The system of claim 6, wherein the memory stores further data for processing by the processor that, when processed, causes the processor to:
generate a notification when at least one of movement of the target and a change to the target is detected.

11. The system of claim 6, wherein the target is within a field of view of the imaging device and the tool is not within the field of view.

12. The system of claim 6, wherein the target is within a field of view of the imaging device and the tool is within the field of view.

13. The system of claim 6, wherein the memory stores further data for processing by the processor that, when processed, causes the processor to:
generate a notification when at least a portion of the target is no longer within a field of view.

14. A system for performing one or more surgical tasks comprising:
a first robotic arm configured to orient a first tool;
a second robotic arm configured to orient a second tool;
at least one processor; and
a memory storing data for processing by the processor, the data, when processed, causing the processor to:
co-registering the first robotic arm and the second robotic arm;
cause the first robotic arm to perform a first task using the first tool; and
cause the second robotic arm to perform a second task using the second tool.

15. The system of claim 14. wherein the second task is dependent on the first task.

16. The system of claim 14, wherein the second task is independent of the first task.

17. The system of claim 14, wherein the first task is performed on a first anatomical element and the second task is performed on a second anatomical element.

18. The system of claim 14, wherein the first task and the second task is performed on an anatomical element.

19. The system of claim 14, wherein the first tool is the same as the second tool.

20. The system of claim 14, wherein the first tool is different than the second tool.
